(19) United States
(12) Patent Application Publication
(10) Pub. No.: US 2008/0097569 A1
(43) Pub. Date: Apr. 24, 2008

(54) REDUCTION OF BURST RELEASE FROM THERAPEUTICALLY TREATED MEDICAL DEVICES

(75) Inventors: Tim O'Connor, County Galway (IE); Aiden Flanagan, County Galway (IE); Dave McMorrow, Galway City (IE); Anthony Malone, County Galway (IE)

Correspondence Address: KENYON & KENYON LLP, 1500 K STREET N.W., SUITE 700, WASHINGTON, DC, 20005, US

(73) Assignee: BOSTON SCIENTIFIC SCIMED, INC., Maple Grove, MN (US)

(21) Appl. No.: 11/857,849

(22) Filed: Sep. 19, 2007

Related U.S. Application Data
(60) Provisional application No. 60/852,978, filed on Oct. 20, 2006.

(51) Int. Cl. A61F 2/06

(52) U.S. Cl. 623/1.2; 427/2.25; 600/36; 623/1.42; 623/1.46

(57) ABSTRACT

The present invention generally relates to the conditioning of coated medical devices such as stents. More specifically, the present invention relates to methods for positioning a medical device within an elution media for a predetermined time period to eliminate a burst release from the coating. Under methods and processes of the invention, a medical device target surface may be identified and coated with therapeutic. The coated surface of the medical device may then be positioned within an elution media for a predetermined period of time to release a predetermined amount of coating.

### RELATED APPLICATIONS

This application claim benefit of 60/852,978, filed Oct. 20, 2006, which is incorporated herein in its entirety.

### TECHNICAL FIELD

The present invention generally relates to the conditioning of therapeutically treated medical devices. More specifically, the present invention relates to conditioning a medical device treated with a therapeutic in order to remove some of the therapeutic prior to placing the medical device within a patient, thereby reducing burst release of therapeutic from the medical device.

### BACKGROUND

The positioning and deployment of medical devices within a target site of a patient is a common, often-repeated procedure of contemporary medicine. The devices or implants that may be employed during these procedures may be used for many medical purposes, including the reinforcement of recently re-enlarged lumens, the replacement of ruptured vessels, and the treatment of disease, such as vascular disease, by local pharmacotherapy (i.e., delivering therapeutic drug doses to target tissues while minimizing systemic side effects). These procedures may be carried out in various places within the body lumina, including: the coronary vasculature; the esophagus; the trachea; the colon; the biliary tract; the urinary tract; the prostate; the brain; and other organs.

Coatings may be applied to the surfaces of these medical devices. These coatings may reduce the trauma suffered during the insertion procedure, facilitate the acceptance of a medical implant into the target site, and improve the post-procedure effectiveness of the implant. Coating the medical devices may also provide for the localized delivery of therapeutic agents to target locations within the body. Such localized drug delivery may avoid the problems of systemic drug administration, e.g., producing unwanted effects on parts of the body which are not to be conditioned and not being able to deliver a high enough concentration of therapeutic agent to the afflicted part of the body.

### BRIEF DESCRIPTION

The present invention is directed to methods, processes, and systems for reducing burst release of therapeutic from medical devices treated with therapeutic. This reduction may be accomplished by dipping a medical device, previously treated with a therapeutic, into an elution media. It may be accomplished by other methods as well. When using elution media, the device may remain in the elution media for a portion of time or until a certain percentage of the therapeutic has left the medical device. This reduction in therapeutic can have the effect of reducing spikes or bursts of therapeutic from eluting from the medical device when the device is initially placed at a target site.

In one of many embodiments, for example, some or all of the outer surfaces of a medical implant may be coated or otherwise interfaced with therapeutic. Now carrying the therapeutic, the implant may then be positioned within an elution media for a period of time such that a portion of the therapeutic from the medical device will be released. In some instances, this dipping may occur for a selected period of time based upon the therapeutic, the medical device, the targeted use, or combinations of these factors. After being conditioned, the medical implant may then be positioned within a target area of a patient where remaining therapeutic may be released from the implant to the target area.

While an implant is discussed above, the medical device may be of various designs. Moreover, these medical devices may carry the therapeutic in a porous matrix that forms the device, they may also contain a coating that also carries the therapeutic. In other words, in some cases the medical device may have a porous region containing therapeutic and it may also have a coating to transport therapeutic, both of which may be pretreated to reduce burst release.

The invention may be embodied in numerous devices and through numerous methods and systems. The following detailed description, which, when taken in conjunction with the annexed drawings, discloses examples of the invention. Other embodiments, which incorporate some or all of the features as taught herein may also be used in accord with the present invention.

### BRIEF DESCRIPTION OF THE DRAWINGS

Referring to the drawings, which form a part of this disclosure:

FIG. 1 shows a method for conditioning a medical device that may be employed in accord with the invention;

FIG. 2 shows an apparatus for applying therapeutic to a medical device that may be employed in accord with the invention;

FIG. 3 shows a stent positioned in an elution media in accord with the invention;

FIG. 4 shows a method for conditioning a stent in accord with the invention;

FIG. 5 shows a non-porous stent in accord with the invention;

FIG. 6a shows a stent comprised of a porous matrix as employed in accord with the invention;

FIG. 6b shows a stent having first and second porous matrix regions as may be employed in accord with the invention;

FIG. 6c shows a stent having porous matrix layers as may be employed in accord with the invention;

FIG. 7 shows a treatment chamber for coating and drying a stent as may be employed in accord with the invention; and

FIG. 8 shows a stent positioned on a delivery device as may be employed in accord with the invention.

### DETAILED DESCRIPTION

The present invention relates to medical devices covered, treated with or otherwise capable of transporting therapeutic. In accord with the invention, the burst release of therapeutic from these devices may be reduced or otherwise controlled by conditioning the device prior to its use at a target site. This conditioning may occur just prior to the performance of the medical procedure as well as during the manufacture or assembly of the medical device. It may occur at other times as well. The conditioning may include placing the therapeutic laden medical device in an elution media to allow some therapeutic to leave the device and then removing the device from the elution media. The amount of time the device remains in the elution media may be predetermined through prior testing and monitoring. The amount of time the device remains in the elution media may be determined by other means as well. By conditioning the device in this fashion a more even and sustainable release of therapeutic from the device may occur when the device is positioned at a target site.

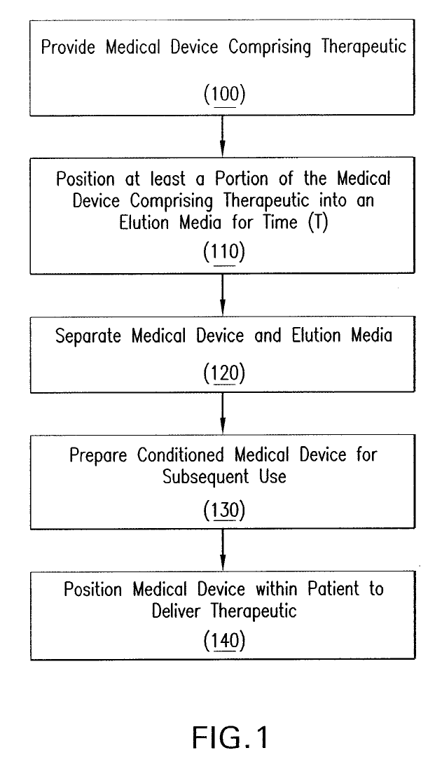

Referring initially to FIG. 1, an exemplary method for conditioning a medical device is shown. This method may include some or all of the steps identified in the figure. It may include other steps, as well as modifications to the identified steps. As identified at 100 the method may include providing a medical device comprising a therapeutic. This medical device may be a catheter, a stent, an aneurism coil and a vast selection of other devices. This device may be laden with a therapeutic on a coating of the device as well as in the device itself. As identified at 110, the method may also include interfacing or dipping some or all of the device into an elution media in order to extract some of the therapeutic from the device. The device may then be removed from the elution media and prepared for subsequent use as shown at 120 and 130 of FIG. 1. Preparing the device for subsequent use may include placing it on another device for use in a patient. Alternatively, as seen in 140, the conditioned device may also be simply placed into a patient to deliver therapeutic.

The medical device described above and throughout the specification may be coated with therapeutic by methods that include dipping, spraying, rolling, brushing, electrostatic plating, vapor deposition, and/or injection. Moreover, in addition to this coating step and the elution burst release conditioning described throughout, other coating steps may also be performed before and after the device is conditioned. For example, after the device is conditioned, one or more surfaces of the medical device may be coated again prior to the device's use.

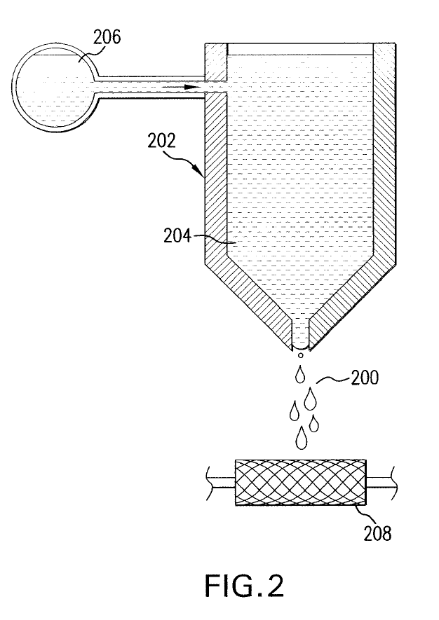

The coating described herein may be carried out with the coating system shown in FIG. 2. In FIG. 2, therapeutic coating 200 may be ejected using a nozzle 202 having a chamber 204 in fluid communication with a coating reservoir 206. A target surface of the medical device 208 may be positioned at a suitable distance from the nozzle 202 and the medical device may be coated with therapeutic and coating 200. Other configurations may be used as well.

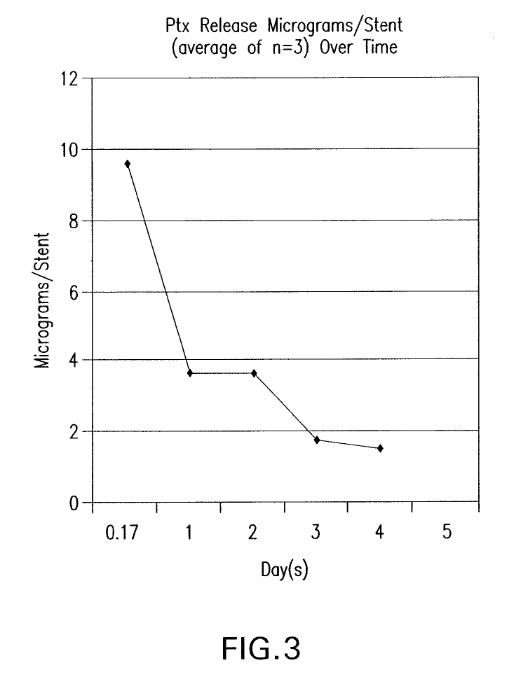

The amount of time that the therapeutically treated medical device will remain in the elution media may be determined by quantitative methods. For example, a burst release curve may be developed using data compiled from testing previously treated medical devices placed in an elution media. In this case and as seen in FIG. 3, an exemplary average burst release curve may be derived from previous testing. In this case, three stents are coated with paclitaxel. The stents may be dipped in the elution media for a period of time and the remaining and/or released therapeutic may be measured. This data may then be graphed. The x-axis may be graphed to illustrate the time period (T), in days, that the medical implants may be immersed in a elution media. The y-axis may be graphed to illustrate the average amount of paclitaxel released, in micrograms/stent, over the time period (T). From this empirical data the amount of time needed for future conditioning to reduce burst release may be determined.

In the example of FIG. 3, a nano-porous stent with a carbon outer surface layer was used and the curve was generated by plotting the data compiled via measuring therapeutic release by techniques employing high performance liquid chromatography. In the example illustrated, the coated stents were positioned in the drug elution media for at least a period of five days to simulate in-vivo conditions. As can be seen by the curve, if a stent were removed from the elution media after the first day and used in-vivo, it would produce a lower sustained release similar to that shown by the remaining part of the curve. Therefore, as seen, potential toxic burst releases, can be reduced, if not eliminated by conditioning the stent prior to use.

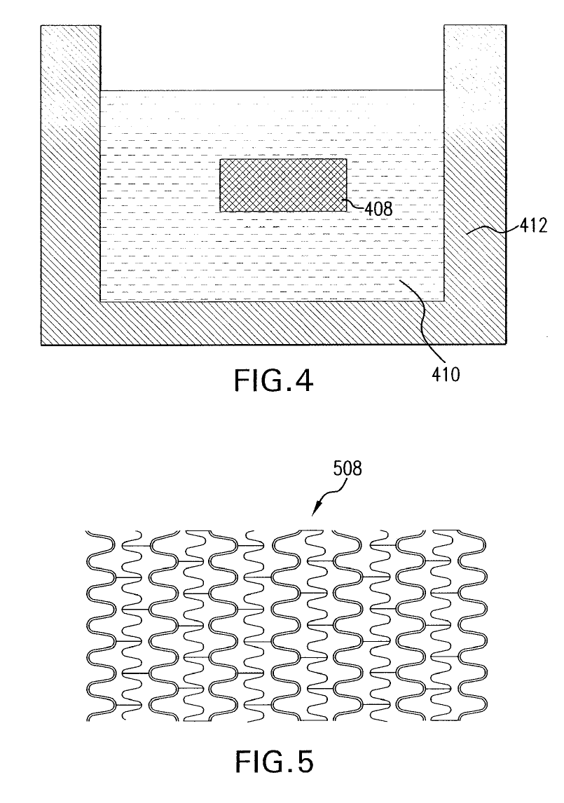

Once the desired burst release time period (T) is determined for the therapeutic this elution time may be used to condition the devices. FIG. 4 shows a system for conditioning these devices. As shown, one or more medical implants 408 may be positioned in an elution media 410 contained in an elution chamber 412 for the time period (T). Any suitable elution media 410 may be used.

For example, a suitable elution media 410 may be an aqueous based media, such as a PBS Tween solution similar to the solution that is used in a standard Kinetic Drug Release test. An alcohol based media may also be used.

The elution media may remove therapeutic, such as paclitaxel, through dissolution from a therapeutic/polymer coating. For instance, the elution media may first dissolve the outermost layer of coating. As the outer layers of the coating are removed, a network of voids are exposed to allow the elution media to dissolve the therapeutic located deeper within the coating. The duration of time that the coating is exposed to the media may determine the amount of therapeutic removed from the coating. The aqueous and alcohol based solutions act in a similar manner, however, the alcohol based elution media (e.g. a 30% IPA alcohol based elution media) may be more aggressive. For example, the alcohol solution may be able to penetrate deeper into the polymer matrix to dissolve the therapeutic from deep within the coating. In some instances, the elution media may actually swell the coating to allow for an elevated level of release.

A gaseous elution media, such as oxygen (O), may also be used to remove coating from the medical device. Other inert gases such as helium (He) and argon (Ar) are also suitable. In this case, the gas may use sublimation or plasma treatment to remove coating. For example, ionized gas, in a relatively strong vacuum environment, may be used to bombard the coating surface to remove weak bonds and typical CH-based organic contamination. For more aggressive treatments, etching, ablation, and tetrafluoromethane (CF4) are also suitable alternatives.

This conditioning may also be conducted at numerous temperatures and over temperature ranges, for example, a temperature around 37 degrees ° C. may be used and a temperature gradient may be applied as well with the elution beginning at one temperature and continuing as the temperature is varied.

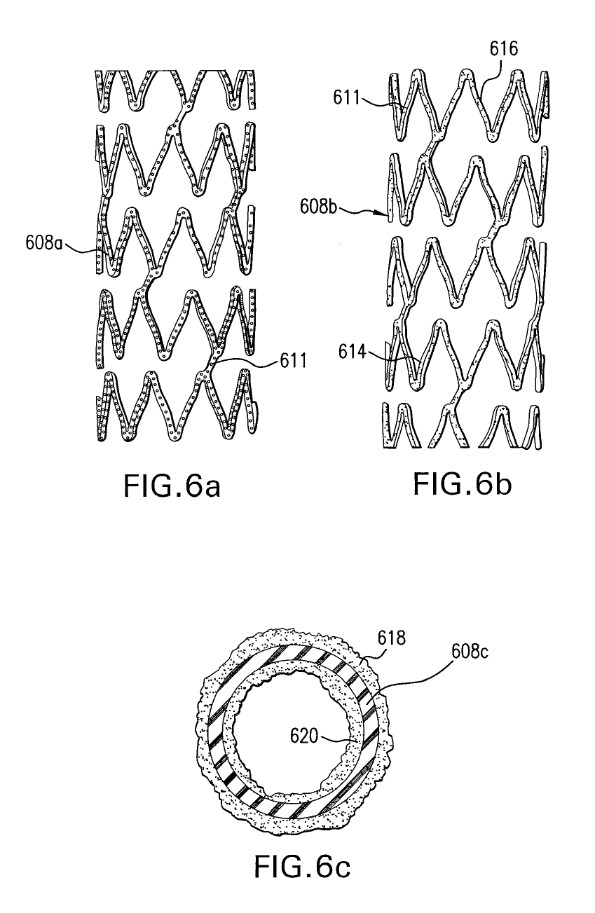

Devices that may be treated as described herein are shown in FIGS. 5 and 6a-c. In FIG. 5, a non-porous stent 508 is shown while in FIG. 6a-6b porous stents 608a, 608b are shown which include pores 611. Stent 608b comprises two porous matrix regions 614 and 616. The first porous matrix region 614 may be characterized by a first porosity and first mean pore size configured to receive different quantities and types of therapeutic while the second porous matrix region 616 may be characterized by a second porosity and a second mean pore size configured to receive different quantities and types of therapeutic. One therapeutic may be loaded into the pores 611 of the first porous matrix region 614 while a second therapeutic may be loaded into the pores 611 of the second porous matrix region 616. The same therapeutic may also be loaded into both the first and the second porous matrix regions 614 and 616.

The medical implant may also be formed of a porous material, and the medical implant may have a porous layer or layers deposited thereon. As seen in FIG. 6c, the stent 608c may have first and second porous layers 618 and 620. The first porous layer 618 may be located on the outside surface of the stent 618 while the second porous layer 620 may be located on the inside surface of the stent 608c. Also, multiple layers may be placed on top of one another and other surfaces of the stent may have a layer deposited thereon. In each of these cases the therapeutic may be conditioned to reduce burst release as described herein.

Medical implants that embody the invention may be used for innumerable medical purposes, including the reinforcement of recently re-enlarged lumens, the replacement of ruptured vessels, and the treatment of disease such as vascular disease by local pharmacotherapy, i.e., delivering therapeutic drug doses to target tissues while minimizing systemic side effects. Examples of such medical implants include stents, stent grafts, vascular grafts, intraluminal paving systems, and other devices used in connection with drug-loaded polymer coatings. Such medical devices are implanted or otherwise utilized in body lumina and organs such as the coronary vasculature, esophagus, trachea, colon, biliary tract, urinary tract, prostate, brain, and the like.

The medical implants themselves may be self-expanding, mechanically expandable, or hybrid implants which may have both self-expanding and mechanically expandable characteristics. The medical implant may be made in a wide variety of designs and configurations, and may be made from a variety of materials including plastics and metals. Additionally, the medical implant may be fabricated from various materials including conductive materials, such as conductive ceramic, polymeric, metallic materials.

Porous medical implants may be made from a powdered material such as powdered metal or polymer. The medical implants of the present invention may be formed of any therapeutic-compatible powdered metals such as stainless steel. Other suitable metals include, but are not limited to, spring steel, nitinol and titanium as well as any other therapeutic-compatible metal which may become available in powdered form in the future. Suitable metals do not produce toxic reactions or act as carcinogens. The medical implants of the present invention may also be prepared with different pore sizes and may be prepared in a range of porosities allowing for the production of stents with differing drug delivery characteristics.

Methods employed with the present invention may also be used with polymer based drug eluting stent coatings. The stent of the present invention may also be formed of therapeutic-compatible powdered polymeric materials such as PTFE.

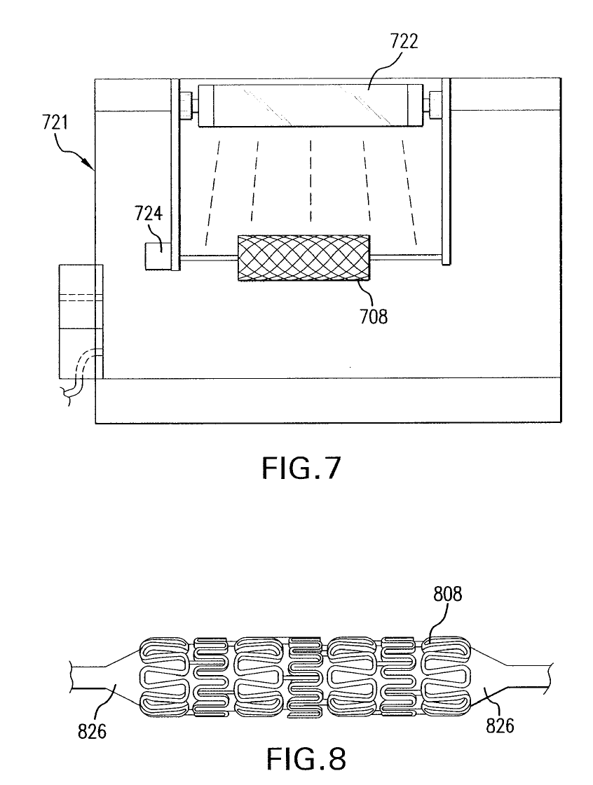

A further step that may be employed with the present invention is the step of removing the medical implant from a elution media and drying the medical implant. The drying step may include the step of applying heat or compressible fluid to the medical implant to facilitate drying. For example, as seen in FIG. 7, the medical implant 708 may be exposed to a coating dryer 722, such as an infrared heater or convection oven, which may be arranged inside of a treatment chamber 721. The medical implant may also be rotated within the treatment chamber 721 via a rotating member 724 to facilitate drying.

The term “treatment chamber” as used herein may be any vessel having defined walls with inside surfaces. A treatment chamber may be made from various materials including clear, translucent, and opaque polymers, metals, and ceramics. Clear polymers, which provide for the internal viewing of implants being coated or impregnated with therapeutics in the treatment chamber 721, may be used in an exemplary embodiment.

The treatment chamber 721 may be preferably cylindrical but it may be other shapes as well. These shapes may include octagons, other multi-sided polygons, ovals, and non-symmetrical shapes. Furthermore, the treatment chamber may be sized to hold one or more implants.

Still another step that may be employed with the embodiments of the present invention is positioning the medical implant on a delivery device and positioning the medical implant within a body. Various methods may be employed for delivery and implantation of the medical implant. For instance, as seen in FIG. 8, a medical implant 808, such as a mechanically expanding stent may be positioned on an expandable member 826, such as a dilatation balloon provided on the distal end of an intravascular catheter, advancing the catheter through a patient's vasculature to the desired location within the patient's body lumen, and inflating the balloon on the catheter to expand the medical implant 808 into a permanent expanded condition.

One method of inflating the expandable member 826 may include the use of inflation fluid. The expandable member may then be deflated and the catheter removed from the body lumen, leaving the medical implant 808 in the vessel to hold the vessel open.

Another suitable method may include the use of self-expanding medical implants that may be typically held in an unexpanded state during delivery using a variety of methods including sheaths or sleeves which cover all or a portion of the medical implant. When the medical implant is in its desired location of the targeted vessel the sheath or sleeve is retracted to expose the medical implant which then self-expands upon retraction.

While various embodiments have been described, other embodiments are plausible. It should be understood that the foregoing descriptions of various examples of the conditioning method are not intended to be limiting, and any number of modifications, combinations, and alternatives of the examples may be employed to facilitate the effectiveness of the controlled release of therapeutic.

The coating, in accord with the embodiments of the present invention, may comprise a polymeric and or therapeutic agent formed, for example, by admixing a drug agent with a liquid polymer, in the absence of a solvent, to form a liquid polymer/drug agent mixture. A suitable list of drugs and/or polymer combinations is listed below. The term “therapeutic agent” as used herein includes one or more “therapeutic agents” or “drugs.” The terms “therapeutic agents” or “drugs” can be used interchangeably herein and include pharmaceutically active compounds, nucleic acids with and without carrier vectors such as lipids, compacting agents (such as histones), viruses (such as adenovirus, adenoassociated virus, retrovirus, lentivirus and α-virus), polymers, hyaluronic acid, proteins, cells and the like, with or without targeting sequences.

Specific examples of therapeutic agents used in conjunction with the present invention include, for example, pharmaceutically active compounds, proteins, cells, oligonucleotides, ribozymes, anti-sense oligonucleotides, DNA compacting agents, gene/vector systems (i.e., any vehicle that allows for the uptake and expression of nucleic acids), nucleic acids (including, for example, recombinant nucleic acids; naked DNA, cDNA, RNA; genomic DNA, cDNA or RNA in a non-infectious vector or in a viral vector and which further may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic and anionic polymers and neutral polymers that are selected from a number of types depending on the desired application. Non-limiting examples of virus vectors or vectors derived from viral sources include adenoviral vectors, herpes simplex vectors, papilloma vectors, adeno-associated vectors, retroviral vectors, and the like. Non-limiting examples of biologically active solutes include anti-thrombogenic agents such as heparin, heparin derivatives, urokinase, and PPACK (dextrophenylalanine proline arginine chloromethylketone); antioxidants such as probucol and retinoic acid; angiogenic and anti-angiogenic agents and factors; anti-proliferative agents such as enoxaprin, angiopeptin, rapamycin, angiopeptin, monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, acetyl salicylic acid, and mesalamine; calcium entry blockers such as verapamil, diltiazem and nifedipine; antineoplastic/antiproliferative/anti-mitotic agents such as paclitaxel, 5-fluorouracil, methotrexate, doxorubicin, daunorubicin, cyclosporine, cisplatin, vinblastine, vincristine, epothilones, endostatin, angiostatin and thymidine kinase inhibitors; antimicrobials such as triclosan, cephalosporins, aminoglycosides, and nitrofurantoin; anesthetic agents such as lidocaine, bupivacaine, and ropivacaine; nitric oxide (NO) donors such as linsidomine, molsidomine, L-arginine, NO-protein adducts, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warfarin sodium, Dicumarol, aspirin, prostaglandin inhibitors, platelet inhibitors and tick antiplatelet factors; vascular cell growth promoters such as growth factors, growth factor receptor antagonists, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; survival genes which protect against cell death, such as anti-apoptotic Bcl-2 family factors and Akt kinase; and combinations thereof. Cells can be of human origin (autologous or allogenic) or from an animal source (xenogeneic), genetically engineered if desired to deliver proteins of interest at the insertion site. Any modifications are routinely made by one skilled in the art.

Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides can also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that can be injected, or whose DNA can be incorporated, include without limitation, angiogenic factors and other molecules competent to induce angiogenesis, including acidic and basic fibroblast growth factors, vascular endothelial growth factor, hif-1, epidermal growth factor, transforming growth factor α and β platelet-derived endothelial growth factor, platelet-derived growth factor, tumor necrosis factor α, hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for conditioning malignancies; and combinations thereof. Still other useful factors, which can be provided as polypeptides or as DNA encoding these polypeptides, include monocyte chemoattractant protein (“MCP-1”), and the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMPs are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNAs encoding them.

As stated above, coatings used with the exemplary embodiments of the present invention may comprise a polymeric material/drug agent matrix formed, for example, by admixing a drug agent with a liquid polymer, in the absence of a solvent, to form a liquid polymer/drug agent mixture. Curing of the mixture typically occurs in-situ. To facilitate curing, a cross-linking or curing agent may be added to the mixture prior to application thereof. Addition of the cross-linking or curing agent to the polymer/drug agent liquid mixture must not occur too far in advance of the application of the mixture in order to avoid over-curing of the mixture prior to application thereof. Curing may also occur in-situ by exposing the polymer/drug agent mixture, after application to the luminal surface, to radiation such as ultraviolet radiation or laser light, heat, or by contact with metabolic fluids such as water at the site where the mixture has been applied to the luminal surface. In coating systems employed in conjunction with the present invention, the polymeric material may be either bioabsorbable or biostable. Any of the polymers described herein that may be formulated as a liquid may be used to form the polymer/drug agent mixture.

The polymer used in the exemplary embodiments of the present invention is preferably capable of absorbing a substantial amount of drug solution. When applied as a coating on a medical device in accordance with the present invention, the dry polymer is typically on the order of from about 1 to about 50 microns thick. In the case of a balloon catheter, the thickness is preferably about 1 to 10 microns thick, and more preferably about 2 to 5 microns. Very thin polymer coatings, e.g., of about 0.2-0.3 microns and much thicker coatings, e.g., more than 10 microns, are also possible. It is also within the scope of the present invention to apply multiple layers of polymer coating onto a medical device. Such multiple layers are of the same or different polymer materials.

The polymer of the present invention may be hydrophilic or hydrophobic, and may be selected from the group consisting of polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers.

Coatings from polymer dispersions such as polyurethane dispersions (BAYHYDROL®, etc.) and acrylic latex dispersions are also within the scope of the present invention. The polymer may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment of the invention, the preferred polymer is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyisocyanates such that the devices become instantly lubricious when exposed to body fluids. In another preferred embodiment of the invention, the polymer is a copolymer of polylactic acid and polycaprolactone.

The examples described herein are merely illustrative, as numerous other embodiments may be implemented without departing from the spirit and scope of the exemplary embodiments of the present invention. Moreover, while certain features of the invention may be shown on only certain embodiments or configurations, these features may be exchanged, added, and removed from and between the various embodiments or configurations while remaining within the scope of the invention. Likewise, methods described and disclosed may also be performed in various sequences, with some or all of the disclosed steps being performed in a different order than described while still remaining within the spirit and scope of the present invention.

What is claimed is:

1. A method of conditioning a therapeutic releasing medical device to reduce burst release of therapeutic, the method comprising:
providing a medical device comprising a therapeutic; and
removing therapeutic from the medical device by positioning at least a portion of the medical device comprising therapeutic into an elution media for a period of time prior to placing the medical device into a patient, the elution media acting to remove therapeutic from the medical device when the medical device is in the elution media.

2. The method of claim 1, further comprising separating the medical device and the elution media after a predetermined amount of time has passed.

3. The method of claim 1, further comprising sampling the elution media after the medical device has been placed in the elution media.

4. The method of claim 3, further comprising testing the sampled elution media to determine the concentration of therapeutic in the elution media.

5. The method of claim 4, further comprising sampling the elution media second time and testing the second sample to determine the concentration of therapeutic in the elution media.

6. The method of claim 1, wherein the medical device is a medical implant and wherein after the medical implant is removed from the elution media the medical implant is positioned on a delivery device.

7. The method of claim 1, wherein the therapeutic is polymer free.

8. The method of claim 1, wherein the elution media is alcohol based or gaseous.

9. The method of claim 1, wherein the medical device comprises a porous matrix, the porous matrix laden with therapeutic.

10. The method of claim 1, wherein the medical device comprises a porous coating, the porous coating laden with therapeutic.

11. The method of claim 1, wherein the medical device is a stent.

12. The method of claim 11, wherein the stent is self-expanding.

13. The method of claim 1 1, wherein the stent is mechanically expandable.

14. The method of claim 1, wherein the medical device is positioned in the elution media for a pre-selected portion of time.

15. The method of claim 14, wherein the period of time in which the medical device is in the elution media has been previously determined.

16. The method of claim 1 further comprising selecting the period of time in which the medical device remains in the elution media from a burst release curve.

17. The method of claim 16, wherein the burst release curve charts the average amount of therapeutic removed from two or more stents over a time period (T).

18. The method of claim 17, wherein the time period (T) is in days.

19. The method of claim 1, wherein the medical device is a medical implant and wherein after the medical implant is removed from the elution media the medical implant is positioned within a patient.

20. A therapeutic releasing medical device formed according to a process including:
providing a medical device comprising a therapeutic; and
removing therapeutic from the medical device by positioning at least a portion of the medical device comprising therapeutic into an elution media for a period of time prior to placing the medical device into a patient, the elution media acting to remove therapeutic from the medical device when the medical device is in the elution media.
